SEQUENCE LISTING (55 sequences)

SEQ ID NO: 1 - DNA, 38 bp, Artificial Sequence
  Synthetic Primer
gcckgatgncgrcgynnnnnrcgycttatcmggcctac

SEQ ID NO: 2 - DNA, 126 bp, Artificial Sequence
  Synthetic Primer
tatacmcwaaatmattcgrgttgcaksaaggcggcaasnkagtgaatycccrggagcwtasataastawgtgactgggrtgarcrarcgmagccaacgcasmtgcrryyygaarkaygamgrgkat

SEQ ID NO: 3 - DNA, 27 bp, Artificial Sequence
  Synthetic Primer
gtncngnntttttgttaatncnctata

SEQ ID NO: 4 - DNA, 90 bp, Artificial Sequence
  repeat_unit
ytagagyatttgmcaaaaagacgcaacgtctttttggcgrgcggactgggacagctcgmagagrgcgagtgcaaaacackgagcagggcg

SEQ ID NO: 5 - DNA, 20 bp, Artificial Sequence
  Synthetic Primer
cctgcgagcgtaggcgtcgg

SEQ ID NO: 6 - DNA, 20 bp, Artificial Sequence
  Synthetic Primer
ctcgtccagcgccgcttcgg

SEQ ID NO: 7 - DNA, 22 bp, Artificial Sequence
  Synthetic Primer
ctacggcaaggcgacgctgacg

SEQ ID NO: 8 - DNA, 18 bp, Artificial Sequence
  Synthetic Primer
nnnncgncgncatcnggc

SEQ ID NO: 9 - DNA, 18 bp, Artificial Sequence
  Synthetic Primer
ncgncttatcnggcctac

SEQ ID NO: 10 - DNA, 30 bp, Artificial Sequence
  Synthetic Primer
ggtgattgccgcgtggttccgttgaatgag

SEQ ID NO: 11 - DNA, 34 bp, Artificial Sequence
  Synthetic Primer
gggggcartarggcagtatgttaagggaataggg

SEQ ID NO: 12 - DNA, 18 bp, Artificial Sequence
  Synthetic Primer
nnnncgncgncatcnggc

SEQ ID NO: 13 - DNA, 18 bp, Artificial Sequence
  Synthetic Primer
ncgncttatcnggcctac

SEQ ID NO: 14 - DNA, 18 bp, Artificial Sequence
  Synthetic Primer
nnnncgncgncatcnggc

SEQ ID NO: 15 - DNA, 18 bp, Artificial Sequence
  Synthetic Primer
ncgncttatcnggcctac

SEQ ID NO: 16 - DNA, 22 bp, Artificial Sequence
  Synthetic Primer
ctacggcaaggcgacgctgacg

SEQ ID NO: 17 - DNA, 22 bp, Artificial Sequence
  Synthetic Primer
acgtggtttgaagagattttcg

SEQ ID NO: 18 - DNA, 22 bp, Artificial Sequence
  Synthetic Primer
atgtaagctcctggggattcac

SEQ ID NO: 19 - DNA, 22 bp, Artificial Sequence
  Synthetic Primer
aagtaagtgactggggtgagcg

SEQ ID NO: 20 - DNA, 20 bp, Artificial Sequence
  Synthetic Primer
tcgctcaaaacaacgacacc

SEQ ID NO: 21 - DNA, 28 bp, Artificial Sequence
  Synthetic Primer
gagtctccggacatgccggggcggttca

SEQ ID NO: 22 - DNA, 15 bp, Artificial Sequence
  Synthetic Primer
gtggtggtggtggtg

SEQ ID NO: 23 - DNA, 21 bp, Artificial Sequence
  Synthetic Primer
catctgtggtggaaagtaaac

SEQ ID NO: 24 - DNA, 21 bp, Artificial Sequence
  Synthetic Primer
ataatgctcaaaggtggtaag

SEQ ID NO: 25 - DNA, 21 bp, Artificial Sequence
  Synthetic Primer
gccgttttggccatagttaag

SEQ ID NO: 26 - DNA, 14 bp, Artificial Sequence
  Synthetic Primer
gttaattcactata

SEQ ID NO: 27 - DNA, 18 bp, Artificial Sequence
  Synthetic Primer
gcggactgggacagctcg

SEQ ID NO: 28 - DNA, 18 bp, Artificial Sequence
  Synthetic Primer
cgagctgtcccagtccgc

SEQ ID NO: 29 - DNA, 18 bp, Artificial Sequence
  Synthetic Primer
attaacaaaaaccggtac

SEQ ID NO: 30 - DNA, 18 bp, Artificial Sequence
  Synthetic Primer
ttttgttaattcactata

SEQ ID NO: 31 - DNA, 22 bp, Artificial Sequence
  Synthetic Primer
ttcgtcagttctatctacaacc

SEQ ID NO: 32 - DNA, 22 bp, Artificial Sequence
  Synthetic Primer
tgcggctagcttcctagtttgc

SEQ ID NO: 33 - DNA, 14 bp, Artificial Sequence
  Synthetic Primer
acaaaaaccggtac

SEQ ID NO: 34 - DNA, 24 bp, Artificial Sequence
  Synthetic Primer
ccgccgttgccgccgttgccgccg

SEQ ID NO: 35 - DNA, 15 bp, Artificial Sequence
  Synthetic Primer
tgtaggccggataag

SEQ ID NO: 36 - DNA, 18 bp, Artificial Sequence
  Synthetic Primer
cgcttaggcctgtgtcca

SEQ ID NO: 37 - DNA, 17 bp, Artificial Sequence
  Synthetic Primer
cacttagccgcgtgtcc

SEQ ID NO: 38 - DNA, 21 bp, Artificial Sequence
  Synthetic Primer
tacattcgaggacccctaagt

SEQ ID NO: 39 - DNA, 25 bp, Artificial Sequence
  Synthetic Primer
aattctaatacgactcactataggg

SEQ ID NO: 40 - DNA, 22 bp, Artificial Sequence
  Synthetic Primer
aattctaatacgactcactata

SEQ ID NO: 41 - DNA, 47 bp, Artificial Sequence
  Synthetic Primer
aattctaatacgactcactatagggctacggcaaggcgacgctgacg

SEQ ID NO: 42 - DNA, 43 bp, Artificial Sequence
  Synthetic Primer
aattctaatacgactcactatagggnnnncgncgncatcnggc

SEQ ID NO: 43 - DNA, 44 bp, Artificial Sequence
  Synthetic Primer
aattctaatacgactcactatactacggcaaggcgacgctgacg

SEQ ID NO: 44 - DNA, 40 bp, Artificial Sequence
  Synthetic Primer
aattctaatacgactcactatannnncgncgncatcnggc

SEQ ID NO: 45 - DNA, 47 bp, Artificial Sequence
  Synthetic Primer
aattctaatacgactcactatagggaagtaagtgactggggtgagcg

SEQ ID NO: 46 - DNA, 47 bp, Artificial Sequence
  Synthetic Primer
aattctaatacgactcactatagggttcattcactgaccccactcgc

SEQ ID NO: 47 - DNA, 47 bp, Artificial Sequence
  Synthetic Primer
aattctaatacgactcactataggggcgagtggggtcagtgaatgaa

SEQ ID NO: 48 - DNA, 47 bp, Artificial Sequence
  Synthetic Primer
aattctaatacgactcactatagggcgctcaccccagtcacttactt

SEQ ID NO: 49 - DNA, 44 bp, Artificial Sequence
  Synthetic Primer
aattctaatacgactcactatagcgagtggggtcagtgaatgaa

SEQ ID NO: 50 - DNA, 45 bp, Artificial Sequence
  Synthetic Primer
aattctaatacgactcactatagggtcgctcaaaacaacgacacc

SEQ ID NO: 51 - DNA, 45 bp, Artificial Sequence
  Synthetic Primer
aattctaatacgactcactatagggagcgagttttgttgctgtgg

SEQ ID NO: 52 - DNA, 45 bp, Artificial Sequence
  Synthetic Primer
aattctaatacgactcactatagggccacagcaacaaaactcgct

SEQ ID NO: 53 - DNA, 45 bp, Artificial Sequence
  Synthetic Primer
aattctaatacgactcactatagggggtgtcgttgttttgagcga

SEQ ID NO: 54 - DNA, 22 bp, Artificial Sequence
  Synthetic Primer
ttcattcactgaccccactcgc

SEQ ID NO: 55 - DNA, 20 bp, Artificial Sequence
  Synthetic Primer
agcgagttttgttgctgtgg

(19) United States
(12) Patent Application Publication
(10) Pub. No.: US 2010/0173279 A1
(43) Pub. Date: Jul. 8, 2010

(54) DETECTION USING PRIMERS TO REPETITIVE DNA AND TRANSCRIPTION-BASED AMPLIFICATION THEREBY

(76) Inventor: Mimi Healy, Gainesville, GA (US)

Correspondence Address: FULBRIGHT & JAWORSKI, LLP, 1301 MCKINNEY, SUITE 5100, HOUSTON, TX, 77010-3095, US

(21) Appl. No.: 11/719,315

(22) PCT Filed: Nov. 14, 2005
(86) PCT No.: PCT/US05/41313

§ 371 Date: May 14, 2009

Related U.S. Application Data
(60) Provisional application No. 60/628,062, filed on Nov. 15, 2004.

(51) Int. Cl. C12Q 1/70; C12Q 1/68

(52) U.S. Cl. 435/5; 435/6

(57) ABSTRACT

The present invention concerns identifying organisms based on detecting distinguishing patterns produced following RNA amplification that originates via a DNA template. In particular, the methods and compositions of the invention concern obtaining ds DNA from the organism in question, amplifying at least part of the DNA via RNA molecules from transcription using primers that target repetitive DNA, and detecting a hallmark pattern of the amplified RNA.

### FIELD OF THE INVENTION

The present invention generally concerns the fields of microbiology, molecular biology, diagnostics, and public health. In particular, the invention regards identifying organisms based on characteristic RNA patterns produced upon amplification originating from a DNA template. More particularly, the invention regards identifying bacteria or fungi, for example, using these methods.

### BACKGROUND OF THE INVENTION

The identification of organisms is useful for patient diagnosis and therapy and for the protection of the public health. Detection of organisms based on distinguishing characteristics provides an unambiguous means for such an identification, and a nucleic acid-based parameter for such an identification is ideal for a definitive determination. Although polymerase chain reaction methods are well-known in the art for such detection methods, in alternative embodiments the polymerase chain reaction is advantageously circumvented, such as through amplification by transcription.

### Fingerprinting by PCR Methods

Carlotti et al. (1997) regard fingerprinting of Candida krusei utilizing a PCR-based amplification with a probe particular for a species-specific repetitive sequence CKRS-1. The PCR products were electrophoresed, blotted, and subject to specific probes to detect the amplification products.

Sander et al. (1998) compare a variety of DNA fingerprinting techniques for typing Bartonella isolates, including pulsed-field gel electrophoresis, enterobacterial repetitive intergenic consensus (ERIC)-PCR, repetitive extragenic palindromic (REP) PCR, and arbitrarily-primed PCR, including by comparison of 16S rDNA sequences from a representative strain.

Olivero et al. (2003) concerns amplifying repeat-containing transcribed sequences through a transcriptome fingerprinting scheme for detecting cancer mutations. The entire cell mRNA was converted into short cDNA fragments having an adapter at both ends, followed by PCR amplification of repeat-containing cDNA fragments with an adaptor-specific primer in conjunction with different arbitrary primers including the repeat. The amplified sequences were subject to gel electrophoresis and sequenced.

U.S. Pat. Nos. 5,523,217 and 5,691,136 describe fingerprinting of bacterial strains via “rep-PCR”, which employs repetitive DNA sequence amplification. In particular, outwardly-directed primers capable of hybridizing to repetitive DNA sequences extend from one repetitive sequence to another hybridizable repetitive sequence. The organism is then identified by visualizing characteristic size-separated extension products.

U.S. Pat. No. 6,074,820 and WO 99/51771 describe detection and differentiation of Mycobacterium by direct variant repeat oligotyping. In particular, in vitro amplification, such as by PCR, LCR, or NASBA, of nucleic acids utilizes a pair of primers comprising sequence complementary to a direct repeat sequence of M. tuberculosis such that amplification reaction occurs for one primer in a 5′ direction and for the other primer in a 3′ direction. More in particularly, M. tuberculosis organisms are differentiated based on a hybridization pattern of the amplified products.

WO 00/77260 regards genomic profiling for a complex biological sample for the presence of particular types of organisms. Specifically, multiple probes that hybridize target molecules in a sample are amplified to determine genomic representation and are detected by contacting or comparing the nucleic acid molecules with a detection ensemble that has a minimum genomic derivation of greater than five. In particular, the nucleic acid molecules are not immobilized as size-fractionated in a matrix or on a solid support. Furthermore, the method is used to quantify a target organism in a biological sample by in situ hybridization. In specific embodiments, the primers target for amplification of sequences lying between Alu repeats using Alu-specific primers.

### Transcription-Based Amplification

U.S. Pat. Nos. 5,130,238 and 5,409,818 providing in a single reaction medium components and conditions for generation of a single-stranded DNA, followed by components and conditions to generate a double-stranded DNA, followed by components and conditions for generation of a plurality of copies of the first template, particularly in the form of RNA transcripts. Particular reaction components include a DNA-directed RNA polymerase, an RNA-directed DNA polymerase, a DNA-directed DNA polymerase, and a ribonuclease that degrades the RNA component of an RNA-DNA hybrid. Reaction products are detected by autoradiography.

WO 96/02668 teaches use of a DNA-directed RNA polymerase, such as an E. coli RNA polymerase of a class that synthesizes cellular RNA in a process for amplifying a specific nucleic acid sequence. The embodiments employ reduction in the number of steps and manipulations compared to known transcription-based amplification steps. Detection of the amplification products utilizes ethidium bromide-stained gels and autoradiography.

WO 99/25868 teaches transcription-based amplification of double-stranded DNA targets by providing a primer complementary to one of the strands of the DNA, wherein the primer has RNA polymerase promoter sequence, providing a primer complementary to the other strand, and the appropriate enzymes. In particular, the invention may be used for amplifying small DNA molecules, such as plasmids. Detection of the amplification products employed autoradiography.

U.S. Pat. Nos. 6,251,639; 6,692,918; and 6,686,156 are directed to amplifying polynucleotides using a composite primer, primer extension, and strand displacement. In particular, the methods amplify a polynucleotide sequence complementary to a target sequence by hybridizing a single stranded DNA template having a target sequence with a composite primer including an RNA portion and a 3′ DNA portion; hybridizing a polynucleotide having a termination sequence to a region of the template that is 5′ with respect to hybridization of the composite primer to the template; extending the composite primer with DNA template; cleaving the RNA portion of the annealed composite primer with an enzyme that cleaves RNA from an RNA-DNA, hybrid, thereby allowing another composite primer to hybridize to the template and repeat primer extension by strand displacement; and hybridizing a polynucleotide comprising a promoter and a region that hybridizes to the displaced primer extension product under conditions that allow transcription to occur by RNA polymerase, thereby producing RNA transcripts that are copies of the target sequence. Products are detected by autoradiography and by ethidium bromide-stained PAGE gels.

Kievits et al. (1991) concerns optimized amplification of HIV-1 polynucleotides using NASBA originating with ssRNA or dsDNA templates. Amplified products were detected by autoradiography using a radioactive probe.

Yates et al. (2001) describe nucleic acid sequence-based amplification (NASBA) utilizing a DNA template as the starting molecule, in contrast to the single-stranded targeted RNA starting material for conventional NASBA. The method utilizes a denaturation step to provide melting of the strands and hybridization of the primer to the generated appropriate single strand and other modifications, including α-casein for improving processivity of DNA-processing enzymes. The resulting products were quantitated via standard concentration identification methods.

Voisset et al. (2000) developed amplification of homologous plasmid DNA under non-denaturing conditions when the plasmid copy was at high levels, such as a plasmid copy number of at least 10⁴. It was also not necessary to denature the plasmid DNA, such as by heating at 65° C. The amplification products were detected by a specific colorimetric detection assay.

Thus, there is a need in the art for providing the organism-specificity of repetitive sequence primers with the advantages of amplification in a non-PCR manner, such as by transcription.

### SUMMARY OF THE INVENTION

The present invention concerns transcription-based amplification following preparation of a suitable DNA template using primers that hybridize to repetitive sequences. In a particular aspect of the invention, the methods use one or more repetitive sequence-based primers, wherein at least one primer comprises an RNA polymerase recognition site, alone or in combination with another primer, to bind complementary DNA sites from an organism. Following generation of a DNA template comprising the RNA polymerase recognition site, transcription-based amplification produces multiple RNA molecules, which may be further defined as fragments followed by detection of the RNA. This method may be referred to herein as Repetitive amplification (RAmp). Multiple RNA molecules are generated thereby and are separated, such as by charge, size, secondary structure and/or a combination thereof, and detected. The separated molecules are detected in a sequence-independent manner, such as by agarose gel electrophoresis and visualized using, for example, ethidium bromide staining and uv light. In alternative embodiments, the RNA molecules are separated through microfluidic lab-on-a-chip technology. The resulting pattern or “fingerprint” can be compared to other isolates for similarity and/or to a database with previously characterized isolates for pattern matching and isolate identification at the genus, species, subspecies, and/or strain level.

In one embodiment of the present invention, there is a method of processing a DNA molecule, comprising the steps of providing at least one ds DNA polynucleotide, the polynucleotide comprising two or more repetitive sequences; providing at least a first primer, said first primer comprising sequence that targets at least part of a repetitive sequence, and a DNA-dependent RNA polymerase promoter sequence or the complement thereof, amplifying at least part of the polynucleotide under conditions that produce RNA molecules from at least part of the polynucleotide; and detecting a plurality of the RNA molecules in a sequence-independent manner.

In a specific embodiment, amplifying steps may be further defined as comprising the steps of producing a double stranded DNA polynucleotide wherein one of the strands comprises at least part of one or more of the repetitive sequences and the DNA-dependent RNA polymerase promoter sequence; and polymerizing the RNA molecules with a DNA-dependent RNA polymerase. In a further specific embodiment, the producing step utilizes a DNA-dependent DNA polymerase or a RNA-dependent DNA polymerase.

The method might also further comprise providing a second primer comprising sequence that targets at least part of a repetitive sequence. In a specific embodiment, the second primer further comprises a DNA-dependent RNA polymerase promoter sequence or the complement thereof. The first and second primers may target the same repetitive sequence or may target different repetitive sequences.

The detecting step may be further defined as identifying a distinguishing pattern of said RNA molecules, which may be identified based on their size, their charge, their secondary structure, or a combination thereof. In specific embodiments, the detecting step comprises electrophoresis, microfluidics chip analysis, or a combination thereof.

The method may also further comprise subjecting at least one of the RNA molecules to the following steps: subjecting the RNA molecule to a third primer, wherein the third primer is optionally the same as the first primer, wherein the third primer comprises sequence that targets at least part of a repetitive sequence and that comprises a DNA-dependent RNA polymerase promoter sequence or the complement thereof, subjecting the RNA molecule and the third primer to a RNA-dependent DNA polymerase, thereby producing a RNA/DNA hybrid, wherein the DNA comprises a DNA-dependent RNA polymerase promoter sequence or the complement thereof; removing the RNA from the RNA/DNA hybrid; and producing RNA molecule copies of at least part of the DNA from the DNA-dependent RNA polymerase promoter sequence.

In a specific embodiment, the RNA is removed from the RNA/DNA hybrid by an enzyme, heat, chemical, or a combination thereof, for example. The enzyme may be RNAse H, such as a DNA-linked RNAse H, for example. The RNA is transcribed from the dsDNA, and the DNA-dependent RNA polymerase may comprise T7 RNA polymerase, Thermus Thermostable RNA Polymerase, or a mixture thereof, for example.

In a specific embodiment of the method, at least one dsDNA polynucleotide originates from one or more organisms, and the one or more organisms are identified based on a distinguishing pattern from the RNA molecules. The method may be further defined as determining the genus of the organism, determining the species of the organism, determining the subspecies of the organism, and/or determining the strain of the organism. In another specific embodiment, one or more organisms is selected from the group consisting of bacteria, fungus, parasite, mammal, insect, marine organism, reptile, plant, or virus.

In an additional embodiment of the present invention, there is a method of identifying an organism having two or more repetitive DNA sequences, comprising the steps of providing at least one ds DNA polynucleotide from the organism, wherein the polynucleotide comprises the two or more repetitive sequences; providing at least a first primer that targets one or more repetitive sequences in the polynucleotide; amplifying at least part of the polynucleotide under conditions that produce RNA molecules; and identifying the organism based on a characteristic pattern from the molecules. In a specific embodiment, the amplifying step is further defined as comprising the steps of producing a double stranded DNA polynucleotide comprising at least part of one or more of the repetitive sequences and a DNA-dependent RNA polymerase promoter sequence; and polymerizing the RNA molecules with a DNA-dependent RNA polymerase. The organism may be a fungus, a bacteria, a mammal, an insect, a marine organism; reptile, plant, or virus. The identifying step comprises electrophoresis of the RNA molecules.

In a particular embodiment there is a kit housed in a suitable container, comprising one or more of the following: at least one primer that targets a repetitive sequence; buffer; ribonucleotides; deoxyribonucleotides; RNA-digesting enzyme; DNA-dependent DNA polymerase; RNA-dependent DNA polymerase; and DNA-dependent RNA polymerase, for example. The primer may be further defined as comprising a DNA-dependent RNA polymerase promoter site or the complement thereof. In specific embodiments, the RNA polymerase promoter site is further defined as a T7 RNA polymerase promoter site, Thermus Thermostable RNA Polymerase, or a mixture thereof.

In another embodiment of the present invention, there is a plurality of RNA molecules generated by a method of the present invention. The plurality may be further defined as being indicative of an organism. The plurality may be comprised on a matrix, such as a gel, a chip, an electropherogram, a paper, or a microarray. In additional specific embodiments, the organism is a bacteria or fungus.

In an additional embodiment of the present invention, there is a pattern of RNA molecules indicative of an organism, said organism comprising a DNA polynucleotide having two or more repetitive sequences, wherein the pattern is produced by the separation of the RNA molecules based on their charge, their size, their secondary structure, or a combination thereof, wherein at least the majority of the RNA molecules comprise at least one sequence derived from a repetitive sequence of the organism. A sequence derived from a repetitive sequence of the organism may be located at the 5′ end, the 3′ end, or both. The pattern may be further defined as being identified in a sequence-independent manner. The pattern may be further defined as being comprised in a matrix, such as a gel, a chip, an electropherogram, a paper, or a microarray.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 illustrates an exemplary embodiment of a method of the invention whereby DNA is utilized as a starting template for eventual amplification in a transcription-based manner using primers that complement repetitive sequences in the DNA.

FIGS. 2A-2C show exemplary embodiments of detection of Repetitive amplification (RAmp). In FIG. 2A, shows examples of separation. FIG. 2B shows detection on an agarose gel. FIG. 2C shows detection on a microfluidic chip. FIG. 2C also specifically demonstrates Ramp amplified product fingerprints from several species of yeast and bacteria, each having distinct fingerprints.

FIG. 3 provides one embodiment wherein RAmp amplified products are visualized using agarose gel electrophoresis. Fingerprints from yeast, mold, Gram+bacteria, Gram−bacteria, and mycobacterium isolates have distinct patterns.

FIGS. 4A and 4B demonstrate exemplary RAmp amplified products. In FIG. 4A, products from Candida and Aspergillus were detected using RNA chips, and this shows genus and species discrimination. In addition, RAmp amplified product from Gram+ and Gram−bacteria using DNA chips (FIG. 4B) shows genus, species, and strain discrimination.

FIG. 5 provides an illustration of species identification among Aspergillus organisms and reproducibility of the fingerprint patterns, as isolates were processed from culture to analysis in triplicate.

FIGS. 6A and 6B show DNA chip detection. In FIG. 6A, there is detection between different species of Candida, different subspecies of Candida, and different strains of Candida, and there is identification of C. albicans using a library of known isolate fingerprints (FIG. 6B).

FIGS. 7A and 7B provide representation of DNA chip detection. In this particular embodiment, it is useful for distinguishing between a variety of fungi, including at the subspecies level (FIG. 7A) and identification of species of A. fumigatus using a library of known isolate fingerprints. (FIG. 7B).

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more.

### I. DEFINITIONS

The term “amplified” as used herein refers to having more than one RNA copy of a particular sequence generated by the repeated transcription with a DNA-dependent RNA polymerase of at least part of the sequence of a DNA polynucleotide.

The term “distinguishing pattern” as used herein refers to a pattern of RNA molecules that is indicative for a particular organism. In some embodiments, the pattern may be indicative of the genus of an organism, whereas in other embodiments the pattern is indicative of other taxonomical levels, such as the species of an organism, the subspecies of an organism, and/or the strain of an organism, where applicable. In particular, the distinguishing pattern is a physical pattern of RNA molecules interpreted by any suitable manner. The physical pattern may be generated based on separation of the molecules by size, charge, secondary structure, or a combination thereof. In exemplary embodiments, the pattern is provided by gel electrophoresis (denaturing or non-denaturing); microfluidics analysis on a DNA or RNA chip; fragment separation using a sequencer, or on a microchip. In specific embodiments, the pattern is observed using real-time amplification with pattern imaging of melt curve or unique pattern matching generated by microarray analysis. The pattern may be visualized by any manner, particularly one suited to the method of displaying the RNA molecules, such as, for example, ethidium bromide staining for gel electrophoresis, fluorescence detection for microfluidics analysis, and so forth.

The term “DNA-dependent RNA polymerase” as used herein refers to an RNA polymerase that catalyzes DNA template-directed extension of the 3′-end of an RNA strand one nucleotide at a time; it is usually capable of initiating a chain de novo.

The term “electropherogram” as used herein refers to a computer-generated graphic representation of RAmp patterns generated by fluorescence of amplified fragment vs. time of fragment migration.

The term “fingerprinting” as used herein refers to the fact that each organism has its characteristic pattern of amplification products based on inherent repetitive sequences that can be used for identification, wherein the repetitive sequences are targeted as the beginning step in an RNA-based amplification method. This unique pattern is each organism's genomic fingerprint.

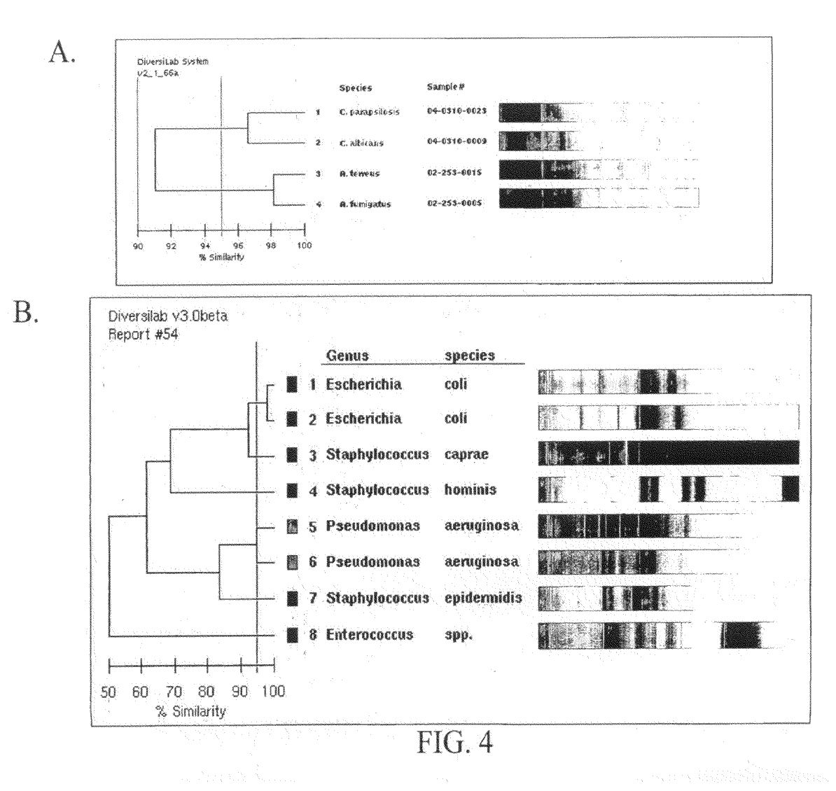

The term “microfluidics chip” as used herein refers to a gel matrix inside a chip having channels etched therein and electrodes for providing a current. A sample of nucleic acid, which may be RNA or DNA, is placed into at least one channel. A fluorescent dye that binds the nucleic acid provides a signal representing the nucleic acid in the channel, and the resultant signal pattern of the RNA or DNA is obtained, which may be referred to as an electropherogram. In other embodiments, software is utilized to produce a gel-like image from the information provided in the electropherogram (see, for example, FIG. 4). A skilled artisan recognizes that RNA microfluidics chips or DNA microfluidics chips are commercially available, and these are distinguished by the different matrix and running conditions. In a particular embodiment, RNA molecules produced by the present invention are distributed on a RNA microfluidics chip, although in other embodiments the RNA is distributed on a DNA microfluidics chip.

The term “pattern” as used herein refers to a reproducible, characteristic composite form of RNA molecules typical of a specific genus, species, sub-species, or strain.

The term “repetitive sequence” as used herein refers to non-coding sequences of DNA containing short repeated sequences and dispersed throughout a genome. The genome may be of any organism, including a prokaryote or eukaryote, and may be a bacteria; a fungus, such as a yeast or mold; a parasite; a mammal; a marine organism; an insect; a virus; a plant; a reptile; and so forth. In particular embodiments, the short repetitive sequences vary from about 30 base pairs to about 500 base pairs. The repetitive sequences are conserved, or similar, in specific embodiments. The term “two or more repetitive sequences” refers to two or more of the same repetitive sequences being present in an organism. In specific embodiments, an organism comprises two or more non-related repetitive sequences.

The term “reverse transcriptase” as used herein refers to a DNA polymerase that can promote the synthesis of DNA using RNA or DNA as a template.

The term “sequence-independent manner” refers to the detection of RNA molecules as described herein wherein a plurality of RNA molecules is detected without using the sequence of the molecules, without knowing the sequence of the molecules, or both. In a particular embodiment, the molecules are detected in the absence of hybridization, including in the absence of identifying one or more of the RNA molecules with a probe, such as a labeled probe, for example.

The term “hybridizes” as used herein refers to at least part of a primer that binds to at least part of a repetitive sequence. A skilled artisan recognizes that a primer may be designed such that it hybridizes at least part of a repetitive sequence of a particular strand, whereas the reverse complement of the primer would hybridize to the respective complementary sequence of the sequence in question. In particular embodiments, the primer is a perfect match to its target sequence, whereas in other embodiments the primer allows minimal mismatch to its target sequence. In particular embodiments, the 3′ end of the primer hybridizes to the repetitive sequence, whereas the 5′ end may or may not. In a specific embodiment, the term “hybridizes” is used interchangeably with the term “binds to,” such as binds to the complement sequence.

The term “T7 RNA polymerase” as used herein refers to a RNA polymerase that can promote the synthesis of RNA using DNA as a template.

### II. Repetitive Sequence Transcription-Based Amplification

The invention generally concerns detection, diagnosis, identification, and/or discrimination of one or more organisms based on fingerprint patterns indicative of the organism. In particular, a RNA pattern specific for the organism in question is produced and identified, such as by comparing to a standard known pattern, for example. This standard known pattern may be provided in any suitable form, such as in an information document accompanying a kit for detection, in a database housing multiple organism patterns, or both, for example. Alternatively, one unknown pattern for a first organism may be compared to another unknown pattern for a second organism for the determination if the first and second organisms are substantially identical or substantially different. The present invention may be used for diagnostic purposes for any organism that comprises one or more repetitive sequences, in specific embodiments.

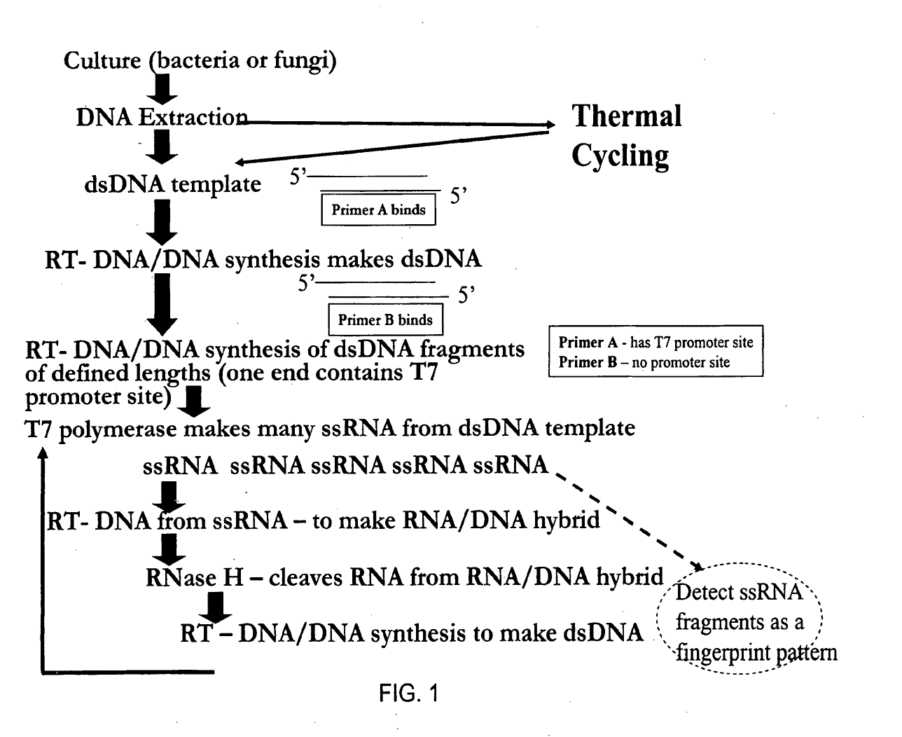

An exemplary embodiment of a method of the present invention is provided in FIG. 1. Briefly, double stranded DNA is obtained from one or more organisms in question, such as by extraction of the DNA. DNA extraction methods are well-known in the art, such as by phenol:chloroform extraction or by commercially available kits, for example. Although the DNA may be extracted, in alternative embodiments the DNA is obtained directly from culture in the absence of extraction, such as from a blood culture. The DNA may be obtained from a single type of organism or from a mixed culture of organisms, if specific primers are used, for example. The one or more organisms may be prokaryotic or eukaryotic, since each has repeat elements. In an alternative method, ssDNA is obtained, such as extracted or derived, from an organism in question and the denaturing step is omitted.

The double stranded DNA is denatured in the presence of at least a first primer (Primer A), although in some embodiments a second primer (Primer B) is also employed. The first primer (Primer A) preferably comprises a DNA-dependent RNA polymerase recognition site, such as a T7 promoter site (which will be hereafter described as an exemplary embodiment only). In an alternative embodiment, Thermus Thermostable RNA Polymerase is employed. A skilled artisan recognizes that Primer A or Primer B can be added together, Primer A can be added first, Primer B can be added first, or Primer A can be added alone, so long as at least one primer has the promoter site recognition sequence. In the presence of a DNA-dependent DNA polymerase or RNA-dependent DNA polymerase, such as reverse transcriptase, and Primer A, a dsDNA strand is produced that comprises a T7 polymerase site on at least one 5′ end of one strand.

T7 polymerase then binds to the corresponding T7 promoter site and transcribes multiple copies of the DNA to RNA. The pool of RNA molecules is utilized as template for one or more rounds of amplification. Specifically, in the presence of the ssRNA, Primer A, and reverse transcriptase, a cDNA-RNA hybrid is generated, after which a reagent for removing the RNA from the hybrid is utilized. Exemplary reagents use RNase H or DNA-linked RNase H (Kanaya et al., 1994) to cleave the RNA from the RNA-DNA hybrid. The single stranded DNA is then targeted by a primer having a repetitive sequence site (Primer B, in exemplary FIG. 1), wherein reverse transcriptase uses a primer to generate dsDNA, followed by T7 RNA polymerase using the dsDNA as template for transcription of RNA molecules from the DNA template. The RNA molecules are then detected by a suitable method and/or are utilized for one or more additional rounds of amplification.

In an alternative embodiment, the above-described method is preceded by addition of Primer A and RT or DNA-dependent DNA polymerase to form dsDNA. The dsDNA is heated to denature the strands, Primer B is added, and the corresponding RNA amplification method proceeds.

### III. Repetitive Sequences and Primers Thereto

One of the puzzles of human evolution has been the much higher percentage of repetitive DNA in humans compared to other invertebrate genomes, wherein the repetitive DNA are stretches of DNA that are not genes but that share the same sequence of base pairs. The repetitive DNA has unknown function. These repetitive elements (which may be referred to as transposable elements) are found so frequently in our genome mainly because they insert into our genome more frequently than they are removed, not because they confer advantage to us. Li et al. (2001) confirmed the very high percentage of repetitive elements in the human genome—on the order of 43 percent, while repetitive elements in the genomes of other diverse organisms, such as Drosophila and Arabidopsis, average about 10 percent. Upon characterizing the location of these elements, particularly the element referred to as Alu, they were found in a surprising number of sequences that encode proteins. The repetitive elements insert into non-coding regions of a gene and are incorporated into protein through alternative splicing by providing splicing sites themselves—places where the editing machinery of the cell cuts genes for translation into proteins—so that new proteins may be created as the coding regions of the old gene are reshuffled, elongated or truncated, for example.

### Interspersed Repetitive Sequences

Intersperesed repetitive sequences comprise copies of transposable elements interspersed throughout the genome, some of which are still active and are often referred to as “jumping genes”. There are at least two classes of interspersed repetitive elements: Class I elements and Class II elements. Class I elements (or “retroelements”—such as retrotransposons, retroviruses, long interspersed nucleotide elements and short interspersed nucleotide elements, for example) transpose via reverse transcription of an RNA intermediate. Class II elements (or DNA transposable elements—such as transposons, Tn elements, insertion sequence elements and mobile gene cassettes of bacterial integrons, for example) transpose directly from one site in the DNA to another. Other terms for interspersed repetitive sequences include repetitive sequences and dispersed interspersed repetitive elements, for example.

### Tandem Repeat Sequences

Tandem repeat sequences refer to copies of DNA sequences that lie adjacent to each other in the same orientation (direct tandem repeats) or in the opposite direction to each other (inverted tandem repeats).

### Terminal Repeat Sequences

Terminal repeat sequences refer to nucleotide sequences that are repeated on both the 5′ and 3′ ends of a particular sequence. For example, some hallmarks of a transposon are that it is flanked by inverted repeats on each end and the inverted repeats are flanked by direct repeats. Examples include the Delta element of Ty retrotransposons and LTRs (long terminal repeats).

Different classes of repetitive DNA elements varying in the size, organization and copy number have been revealed in mammalian genomes. Short and long interspersed repetitive elements (respectively referred to as SINEs and LINEs) are the most abundant. The absence of the obvious specific function of these repeats led to term them as a “selfish DNA.” Recent discoveries demonstrated that they are not a useless part of the genome but may interact with the whole genome and play an important role in its evolution and function, for example (for review see Makalowski, 1995).

As mentioned above, naturally occurring interspersed repetitive DNA elements, found in many (if not all) bacteria, can serve as primer sites for genomic DNA amplification (Versalovic et al. 1991; 1994; de Bruijn, 1992). Several families of repetitive sequences are interspersed throughout the genome of diverse bacterial species (see Lupski and Weinstock 1992). Three exemplary families of repetitive sequences have been studied in most detail, including the 35-40 bp repetitive extragenic palindromic (REP) sequence, the 124-127 bp enterobacterial repetitive intergenic consensus (ERIC) sequence, and the 154 bp BOX element (see Versalovic et al., 1994). These sequences appear to be located in distinct intergenic positions all around the chromosome. The repetitive elements may be present in both orientations on the chromosome, and PCR primers have been designed to “read outward” from the inverted repeats in REP and ERIC, and from the boxA subunit of BOX (Versalovic et al., 1994).

Thus, in particular embodiments, the present invention utilizes repetitive sequences for primer targeting to provide sequences to be amplified via transcription. One or more repetitive sequences may be targeted in a single reaction, in specific embodiments. The repetitive sequence to which the primers bind can be selected from any of the repetitive regions that are present in any organism, including bacteria and fungi, for example, including any types of repetitive regions or combinations thereof.

As described in the examples above, there are many types of repetitive elements in multiple different organisms, and these elements are known in the literature or would be easily discernable using well-known methods in the art, such as by sequencing. For example, for one skilled in the art it would be reasonable to utilize primers that hybridize to a complementary repetitive element and amplify regions within and/or between multiple repetitive element sites, thus producing a distinct RAmp pattern for each. The present invention methods are such that they would work for either a DNA template comprising the repetitive elements or a RNA template comprising the repetitive elements.

Repetitive sequences for any organism may be known in the art or may be determined upon at least partial sequencing of the genome, for example. The repetitive sequences can be identified by a variety of methods. This may be done manually by comparing the sequences of the published nucleic acid sequences for bacterial genomes. A more convenient method, however, is to use a computer program to compare the sequences. In this way one can generate a consensus DNA sequence for use in the methods of the present application.

As used herein, the repetitive sequences may refer to repetitive extragenic palindromic elements. A REP consensus sequence is shown in SEQ ID NO:1 In other embodiments, “ERIC” refers to the enterobacterial repetitive intergenic consensus sequence, which is provided in SEQ TD NO:2. The consensus sequence of “Ngrep,” which refers to the Neisseria repetitive elements, is shown in SEQ ID NO:3. The consensus sequence of “Drrep,” which refers to the Deinococcus repetitive elements, is shown in SEQ ID NO:4. These repetitive elements are found interspersed throughout the bacterial genome, in particular aspects of the invention. In specific embodiments, these four sequences or any combination of these four sequences can be used in the present invention. Further, one skilled in the art will understand that other sequences not provided herein can also be used in the method of the present invention. By binding primers to these repetitive sequences and performing RNA-based amplification, one can generate unique fingerprints and identify individual strains of bacteria.

Other exemplary repetitive sequences are well-known in the art. Repetitive sequences in eubacteria may be targeted, for example, such as those described in Versalovic et al. (1991) (see also Table 1). Particular Mycobacterium tuberculosis repetitive sequences are provided in U.S. Pat. No. 5,370,998, and exemplary primers for targeting thereof include 5′-CCTGCGAGCGTAGGCGTCGG-3′ (SEQ ID NO:5) and 5′-CTCGTCCAGCGCCGCTTCGG-3′ (SEQ ID NO:6). Other embodiments of repetative sequences relate to a Mycobacterium tuberculosis-specific DNA fragment containing IS-like and repetitive sequences, as described in EP 0945462. In specific embodiments, as described therein, a nucleotide sequence of the DNA fragment of that invention that is 1291 bp long or a fragment thereof is directed by repetitive primers. This nucleotide sequence comprises several interesting features including the presence of repeat sequences and an IS-like sequence with an open reading frame. The IS-like sequence is characterized by the presence of two inverted repeats flanked with direct repeat GTT on either side. GTT is a direct repeat which is located at 458 to 460 and at 1193 to 1195. In particular, primers targeting the inverted repeats located at 461 to 469 (TCCGGTGCC) and at 1184 to 1192 (GGCACCGGA) may be utilized in the invention.

Helicobacter pylori repetitive sequences may be targeted using methods of the present invention, such as by utilizing primers similar or identical to those of Go et al. (1994); Go et al. (1995); and Miehlke et al. (1999), for example.

E. coli organisms may be distinguished with methods and compositions of the present invention targeting repetitive DNA sequences in the genome, such as by employing the BOX and REP primers (Dombek et al., 2000), and particular primer sequences that are useful include, for example, BOX AIR (5′-CTACGGCAAGGCGACGCTGACG-3′ (SEQ ID NO:7)) and REP 1R (5′-IIIICGICGICATCIGGC-3′ (SEQ ID NO:8) and REP 2I (5′-ICGICTTATCIGGCCTAC-3′ (SEQ ID NO:9)).

Repetitive sequences have been targeted by primers to classify and differentiate among strains of E. coli (Lipman et al., 1995), Rhizobium meliloti (de Bruijn, 1992), Bradyrhizobium japonicum (Judd et al., 1993), Strepetomyces spp. (Sadowsky et al., 1996), Xanthomonas spp. (Bouzar et al., 1999), and others (Versalovic et al., 1998), and in particular embodiments of the present invention these primers may be employed in the methods of the present invention.

Brucella species (such as B. abortus, B. canis, and B. melitensis) may be strain typed, for example, based on the inventive methods employing primers to known variable known tandom repeats (VNTRs), such as ones comprising “AGGGCAGT” at multiple loci in the genome (Bricker et al., 2003). Exemplary primers utilized therein include LOCUS-1 Fwd (5′-GGTGATTGCCGCGTGGTTCCGTTGAATGAG-3′; SEQ ID NO:10) and REV-3 (5′-GGGGGCARTARGGCAGTATGTTAAGGGAATAGGG-3′; SEQ ID NO: 11).

Salmonella subspecies may be typed using repetitive primers and the methods of the invention. Exemplary repetitive primers for subtyping Salmonella species include those described in Johnson et al. (2001), including ERIC2 and BOXALR primers (see Table 1).

Antonio and Hillier (2003) describe primers that may be employed in methods of the invention, such as those for straintyping of Lactobacillus. Exemplary primers used therein include REP IR-Dt and REP2-Dt (see Table 1).

Exemplary embodiments of fungus detection include targeting the repetitive sequences of Nocardia asteroides with the BOX-AIR primer (Yamamura et al., 2004), for example, using methods of the invention. An additional exemplary fungus having repetitive sequences includes Pythium, such as Pythium ultimum, in the intergenic region of the ribosomal DNA repeat unit. Although length heterogeneity was identified using primers outside this region (Buchko and Klassen, 1990), the repetitive sequences themselves may be targeted pursuant methods of the present invention.

Repetitive sequences are also present in organisms other than bacteria and fungi, such as Leishmania. For example, Schonian et al. (1996) used primers to mini- and microsatellite DNA sequences such as the M13 core sequence and the simple repeat sequences (GTG)₅ and (GACA)₄ to identify relationships of species and strains of Leishmania, such as L. donovani, L. mexicana, and L. braziliensis. Such primers may be used for methods of the present invention.

Other examples of repetitive sequence-targeting primers that may be employed in the invention include those as described in Riley et al. (1991), which were utilized for PCR. As described therein, complex product patterns were demonstrated for a wide variety of eukaryotic microorganisms, including the pathogenic protozoan parasites T. vaginalis, Giardia lamblia, Leishmania donovani, three species of Trypanosoma, and four species of Acanthamoeba; the nonpathogenic protozoans, Paramecium tetraurelia and Tetrahymena thermophilia; and a yeast, Saccharomyces cerevisiae.

Additional repetitive sequences are described in Aranda-Olmedo et al. (2002) regarding species-specific repetitive extragenic palindromic (REP) sequences in Pseudomonas putida, and the exemplary primers described therein or other suitable primers may be employed in the invention.

### IV. Organisms for Detection

The present invention provides diagnostic methods and compositions for detecting one or more particular organisms. The organism may be a prokaryote, a eukaryote, or a mixture thereof. In a specific embodiment, one or more organisms are detected from a mixture of organisms. The organism for detection may be extant or extinct. The organism may be obtained from any type of environment, such as a solid environment, a liquid environment, or a gaseous environment. The organism may be obtained from land, water, or air. The organism may be obtained from a public facility. The organism may be obtained, for example, from a health care facility, such as a hospital or doctor's office; a cafeteria; a restaurant; an earth-orbiting object, such as the space station; an airport; a mall; a theater; an office building; and so forth.

In particular, the organism for detection comprises at least one DNA molecule having two or more repetitive sequences. In particular embodiments, the DNA is suitable for serving as a template for processing in an amplification method. In specific embodiments, the DNA is double stranded, although ssDNA may be obtained and utilized in the inventive methods wherein the denaturing step is not performed. The dsDNA may be a genome, plasmid, mitochondrial DNA, chloroplast DNA, and so forth.

A. Bacteria

The present invention may be used to detect one or more types of bacteria. In a particular embodiment, the detection is of eubacteria (true bacteria; those bacteria having rigid cell walls), although the bacteria for detection may include Archaebacteria (having cell walls, cell membranes, and ribosomal RNA different from those of eubacteria, such as the absence of peptidoglycan, a protein-carbohydrate found in the cell walls of Eubacteria; they are capable of living in harsh environments, such as acidic hot springs, near undersea volcanic vents, and highly salty water). The bacteria may be spherical, rod-shaped, or spiral-shaped; they may be aerobic or anaerobic; and they may be Gram-positive or Gram-negative.

Any source of bacterial nucleic acid in purified or non-purified form can be utilized as starting material, provided it contains or is suspected of containing a bacterial genome of interest. Thus, the bacterial nucleic acids may be obtained from any source that can be contaminated by bacteria. When looking for bacterial infection or in distinguishing bacteria from human or animal subjects, for example, the sample to be tested can be selected or extracted from any bodily sample such as blood, urine, spinal fluid, tissue, vaginal swab, stool, amniotic fluid or buccal mouthwash, for example.

In particular embodiments, the present invention provides a composition that comprises a repetitive DNA segment that is specific for members of the Mycobacterium tuberculosis complex (e.g., M. tuberculosis, M. bovis, and M. bovis BCG). The DNA segment can be used as a hybridization probe and as a target of amplification for the direct detection of the DNA from the Mycobacterium tuberculosis complex in clinical material. In one embodiment of the present invention, DNA segment repeats in the chromosome of M. tuberculosis are targeted by primers. In another embodiment, the targeted nucleotide sequence of the DNA segment is conserved in all copies of the chromosomes of M. tuberculosis complex. For example, as described in U.S. Pat. No. 5,370,998, three cloned DNA segments of M. tuberculosis hybridized with multiple chromosomal fragments of M. tuberculosis complex, indicating the repetitive nature of the DNA segments. Specifically, each segment was found to repeat in the range of about 10-16 times in the M. tuberculosis chromosome. Exemplary Mycobacterium organisms for detection with methods and compositions of the present invention include at least the following: M. smegmatis; M. phlei; M. fortuitum; M. chelonae; M. flavescens; M. chelonae; M. trivale; M. duvali; M. marinum; M. gordonae; M. kansasii; M. avium; M. intracellulare; M. scrofulaceum; M. gordonae; M. xenopi; M. aurum; M. microti; and M. szulgai.

Other bacteria for identification include Escherichia, such as E. coli, E. blattae, E. fergusonii, E. hermannii, and E. vulneris; Bacillus, such as Bacillus parnilus, Bacillus pumilus. Bacillus licheniformi, B. anthracis, B. cereus; Enterococcus, such as VRE spp., Enterococcus faecium, Enterococcus casseliflavus, and Enterococcus gallinarum; Pseudomonas species, such as P. putida, P. aeruginosa, P. cepacia, P. putida, P. stutzeri, P. vesicularis, P. mendocina, and so forth. Particular Staphylococcus species that may be identified as described herein include S. aureus, S. capitis, S. sciuri, and S. lentus, for example.

B. Fungus

The present invention may be employed to detect any fungus so long as the organism comprises nucleic acid having repetitive sequences. Exemplary fungus include Candida, Aspergillus, and Nocardia. Particular Candida species for identification include C. lusitianiae, C. tropicalis, C. parapsilosis, C. albicans, and C. glabrata. Specific Aspergillus species for identification include A. terreus, A. fumigatus, Aspergillus flavus, Aspergillus niger, Aspergillus clavatus, Aspergillus glaucus group, Aspergillus nidulans, Aspergillus oryzae, Aspergillus terreus, Aspergillus ustus, and Aspergillus versicolor.

Additional fungus organisms for use in methods of the present invention include Saccharomyces, such as Saccharomyces cerevisiae,

C. Other

The methods of the present invention may be employed for archaeological purposes, for example, such as for differentiating various species of the genus Homo, including H. sapiens, H. erectus, H. neanderthals, H. ergaster, and H. rudolfensis, for example. Parasites such as Leishmania and Pythium, such as Pythium ultimum, may also be identified with methods of the invention.

The methods of the present invention may also be employed for detecting algae, such as Volvox carteri, for example. Particular sequences to which primers may be directed include those described in Aono et al. (2002), for example. Other Volvox species for detection include V. aureus, V. globactor, V. dissipatrix, and V. tertius, for example.

In particular embodiments of the present invention, the methods of the present invention are useful for distinguishing viruses.

### V. Primers that Target Repetitive DNA

The term “primer,” which may be referred to as “oligonucleotide primer,” as used herein defines a molecule comprised of more than three deoxyribonucleotides. Its exact length will depend on many factors relating to the ultimate function and use of the oligonucleotide primer, including temperature, source of the primer and use of the method. The oligonucleotide primer can occur naturally (as a purified fragment or restriction digestion product) or be produced synthetically, for example. The oligonucleotide primer is capable of acting as an initiation point for synthesis when placed under conditions that induce synthesis of a primer extension product complementary to a nucleic acid strand. The conditions can include the presence of nucleotides and an inducing agent, such as a DNA polymerase, at a suitable temperature and pH. In a particular embodiment, the primer is a single-stranded oligodeoxyribonucleotide of sufficient length to prime the synthesis of an extension product from a specific sequence in the presence of an inducing agent. In a specific embodiment of the present invention, the oligonucleotides are usually between about a 10-mer and 29-mer. In the preferred embodiment they are between about a 15-mer and a 25-mer. Sensitivity and specificity of the oligonucleotide primers are determined by the primer length and uniqueness of sequence within a given sample of a template DNA. Primers that are too short, for example less than about a 10-mer, may show non-specific binding to a wide variety of sequences in the genomic DNA and thus may not be very helpful.

Each primer herein is selected to be substantially complementary to the appropriate strand of each specific repetitive sequence to which the primer binds. For example, for embodiments using two primers, one primer, such as one primer of a pair of primers, is sufficiently complementary to hybridize with a part of the sequence in the sense strand, and the other primer of each pair is sufficiently complementary to hybridize with a different part of the same repetitive sequence in the anti-sense strand. The term “sufficiently complementary” as used herein refers to template-driven polymerization being able to occur from the 3′ end of the primer.

It should also be recognized that a single primer may be utilized alone in this invention, so long as it comprises a T7 recognition site. Because the primer binds to repetitive sequences and because the repetitive sequences can be orientated in both directions, a single primer can bind to both strands of a repetitive sequence and amplify the sequence between two separate repetitive sequences.

At least one primer comprises a T7 polymerase recognition site at the 5′ end, which may be referred to as a tag. Concerning the sequence of the primer, such as particularly for designing the primer, it is known that the sequences in the repetitive elements can be tandem and palindromic. Therefore, it is reasonable that one primer can act both as the forward and the reverse primer. However, in embodiments wherein a T7 tag is present on the 5′ end of the primer, the primer will no longer complement the palindrome. Thus, primers that can be used in the invention include a repeat primer, the complement, the palindrome and the complement to the palindrome with the T7 tag. In specific embodiments, primers with no T7 tag that can be used include those that hybridize to at least part of a repeat sequence and primers that contain a T7 tag that can be used include those that hybridize to at least part of a repeat sequence. In particular, the 3′ end of the primer hybridizes to the respective repetitive sequence, and the 3′ end thus may be designed specifically to facilitate this, such as by targeting GC-rich regions.

In some embodiments, outwardly directed primers may be employed in the invention. As used herein the term “outwardly directed” primer pair refers to the oligonucleotide primers. For example, one primer is substantially complementary to the sense strand and would bind to the sense strand in such an orientation that an extension product generated from the 3′ end of the primer would extend away from the repetitive DNA sequence to which the oligonucleotide primer is bound and across the non-repetitive DNA to a second repetitive DNA sequence. The other member of the primer pair would bind to the antisense strand in an orientation such that an extension product generated on the 3′ end would extend away from the repetitive DNA sequence to which the primer is bound and across the non-repetitive DNA to the next repetitive DNA sequence. Thus, within a specific repetitive DNA sequence the primer pair is bound to the complementary DNA strands 5′ to 5′ and, thus, the extension products grow away from each other across the non-repetitive DNA. The extension products from the two-paired primers are complementary to each other and can serve as templates for further synthesis by binding the other member of the primer pair.

The oligonucleotide primers may be prepared using any suitable method known in the art. For example, the phosphodiester and phosphotriester methods or automated embodiments thereof may be used. It is also possible to use a primer that has been isolated from biological sources, such as with a restriction endonuclease digest.

In particular embodiments, exemplary primers to repetitive sequences are

| | |
|---|---|
| Primer Name | Primer Sequence |

| | | |
|---|---|---|
| REP1R-I | 18 | 5′-III ICG ICG ICA TCI GGC-3′(SEQ ID NO: 12) |
| REP2-I | 18 | 5′-ICG ICT TAT CIG GCC TAC-3′(SEQ ID NO: 13) |
| REP1R-Dt | 18 | 5′-III NCG NCG NCA TCN GGC-3′(SEQ ID NO: 14) |
| REP2-Dt | 18 | 5′-NCG NCT TAT CNG GCC TAC-3′(SEQ ID NO: 15) |
| BOXA1R | 22 | 5′-CTA CGG CAA GGC GAC GCT GAC G-3′(SEQ ID NO: 16) |
| BOXA2R | 22 | 5′-ACG TGG TTT GAA GAG ATT TTC G-3′(SEQ ID NO: 17) |
| ERIC-1R | 22 | 5′-ATG TAA GCT CCT GGG GAT TCA C-3′(SEQ ID NO: 18) |
| ERIC-2 | 22 | 5′-AAG TAA GTG ACT GGG GTG AGC G-3′(SEQ ID NO: 19) |
| RW3A | 23 | 5′-TCG CTC AAA ACA ACG ACA CC-3′(SEQ ID NO: 20) |
| | | 5′-GAG TCT CCG GAC ATG CCG GGG CGG TTC A-3′(SEQ ID |
| | NO: 21) | |
| IR1 | 28 | |
| GTG5 | 15 | 5′-GTG GTG GTG GTG GTG-3′(SEQ ID NO: 22) |
| Ca-21 | 21 | 5′-CAT CTG TGG TGG AAA GTA AAC-3′(SEQ ID NO: 23) |
| Ca-22 | 21 | 5′-ATA ATG CTC AAA GGT GGT AAG-3′(SEQ ID NO: 24) |
| Com-21 | 21 | 5′-GCC GTT TTG GCC ATA GTT AAG-3′(SEQ ID NO: 25) |
| NGREP2 | 14 | 5′-GTT AAT TCA CTA TA-3′(SEQ ID NQ: 26) |
| DRREP1 | 18 | 5′-GCG GAC TGG GAC AGC TCG-3′(SEQ ID NO: 27) |
| DRREP1R | 18 | 5′-CGA GCT GTC CCA GTC CGC-3′(SEQ ID NQ: 28) |
| NGREP1R-18 | 18 | 5′-ATT AAC AAA AAC CGG TAC-3′(SEQ ID NO: 29) |
| NGREP2-18 | 18 | 5′-TTT TGT TAA TTC ACT ATA-3′(SEQ ID NO: 30) |
| BOXB1 | 22 | 5′-TTC GTC AGT TCT ATC TAC AAC C-3′(SEQ ID NO: 31) |
| BOXC1 | 22 | 5′-TGC GGC TAG CTT CCT AGT TTG C-3′(SEQ ID NO: 32) |
| NGREP1R | 14 | 5′-ACA AAA ACC.GGT AG-3′(SEQ ID NO: 33) |
| MBOREP1 | 24 | 5′-CCG CCG TTG CCG CCG TTG CCG CCG-3 (SEQ ID NO: 34) |
| RUPUb1 | 15 | 5′-TGT AGG CCG GAT AAG-3′(SEQ ID NO: 35) |
| IS3A | 15 | 5′-CGC TTA GGC CTG TGT CCA-3′(SEQ ID NO: 36) |
| IS3B | 17 | 5′-CAC TTA GCC GCG TGT CC-3′(SEQ ID NO: 37) |
| BG2 | 22 | 5′-TAC ATT CGA GGA CCC CTA AGT G-3′(SEQ ID NO: 38) |
| T7ggg tag (T7 promoter sequence | 25 | 5′AAT TCT AAT ACG ACT CAC TAT AGG G- 3′(SEQ ID NO: 39) |
| with ggg) | | |
| T7 tag (T7 promoter sequence | 22 | 5′-AAT TCT AAT ACG ACT CAC TAT A-3′(SEQ ID NO: 40) |
| without ggg) | | |
| T7ggg-B1 (the following are all either | 47 | 5′AAT TCT AAT ACG ACT CAC TAT AGG GCT ACG GCA AGG |
| primers or complement or palindrome | | CGA CGC TGA CG-3 (SEQ ID NO:41) |
| of current rep primers with the Tag) | | |
| T7ggg-Dt1 | 43 | 5′-AAT TCT AAT ACG ACT CAC TAT AGG GII INC GNC GNC ATC |
| | | NGG C 3′(SEQ ID NO: 42) |
| T7-B1 | 44 | 5′-AAT TCT AAT ACG ACT CAC TAT ACT ACG GCA AGG CGA |
| | | CGC TGA CG 3′(SEQ ID NO: 43) |
| T7-Dt1 | 40 | 5′-AAT TCT AAT ACG ACT CAC TAT AII INC GNC GNC ATC NGG |
| | | C 3′(SEQ ID NO: 44) |
| T7ggg-E2 | 47 | 5′-AAT TCT AAT ACG ACT CAC TAT AGG GAA GTA AGT GAC |
| | | TGG GGT GAG CG-3′(SEQ ID NO: 45) |
| T7ggg-E2 complement | 47 | 5′-AAT TCT AAT ACG ACT CAC TAT AGG GTT CAT TCA CTG |
| | | ACC CCA CTC GC-3′(SEQ ID NO: 46) |
| T1ggg-E2 palindrome | 47 | 5′-AAT TCT AAT ACG ACT CAC TAT AGG GGC GAG TGG GGT |
| | | CAG TGA ATG AA-3′(SEQ ID NO: 47) |
| T7ggg-E2 palindrome complement | 47 | 5′-AAT TCT AAT ACG ACT CAC TAT AGG GCG CTC ACC CCA |
| | | GTC ACT TAC TT-3′(SEQ ID NO: 48) |
| T7-E2 palindrome | 44 | 5′-AAT TCT AAT ACG ACT CAC TAT AGC GAG TGG GGT CAG |
| | | TGA ATG AA3′(SEQ ID NO: 49) |
| T7ggg-RW3A | 45 | 5′-AAT TCT AAT ACG ACT CAC TAT AGG GTC GCT CAA AAC |
| | | AAC GAC ACC-3 (SEQ ID NO: 50) |
| T7ggg-RW3A complement | 45 | 5′-AAT TCT AAT ACG ACT CAC TAT AGG GAG CGA GTT TTG |
| | | TTG CTG TGG-3′(SEQ ID NO: 51) |
| T7ggg-RW3A palindrome | 45 | 5′-AAT TCT AAT ACG ACT CAC TAT AGG GCC ACA GCA ACA |
| | | AAA CTC GCT-3′(SEQ ID NO: 52) |
| T7ggg-RW3A palindrome | 45 | 5′-AAT TCT AAT ACG ACT CAC TAT AGG GGG TGT CGT TGT |
| complement | | TTT GAG CGA-3′(SEQ ID NO: 53) |
| E2 complement | 22 | 5′-TTC ATT CAC TGA CCC CAC TCG C-3′(SEQ ID NO: 54) |
| RW3A complement | 20 | 5′-AGC GAG TTT TGT TGC TGT GG-3′(SEQ ID NO: 55) |

### VI. Detection of Amplified RNA Products

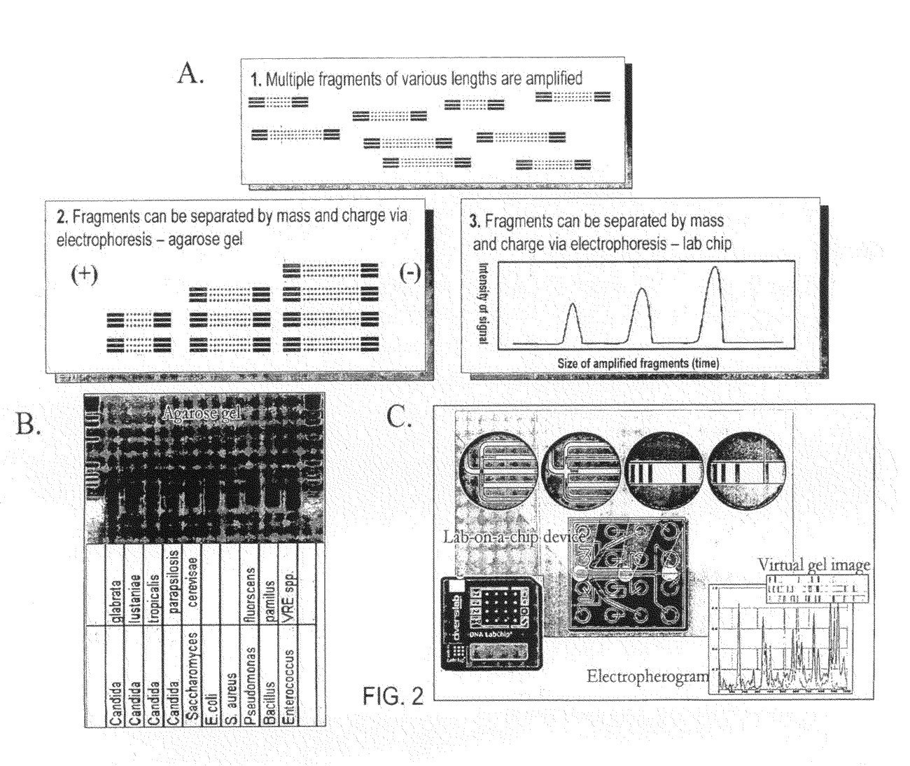

Any fragment separation and detection technology may be utilized for detection of the amplified RNA products, which may be further defined as transcription products, including agarose gels, microfluidic chips, fragment separation on a sequencer, or conformational changes such as real time melt curve analysis or pattern matching on a microarray, for example. FIG. 1 illustrates exemplary embodiments of Ramp, and FIG. 2 illustrates exemplary embodiments of detection of RAmp. During the initiating step of the amplification process, primers that target repetitive sequences bind to at least one, and preferably many, specific repetitive sequences, interspersed throughout the genome. As a result of the amplification process, multiple fragments of various lengths are amplified. These fragments may be employed in any manner to provide a characteristic and distinguishing fingerprint of the organism from which the original template DNA was obtained and RNA molecules amplified thereby (FIG. 2). In specific embodiments, the methods of the present invention avoid probe technology for identification of the RNA molecules and detect the molecules in a sequence-independent manner.

In particular aspects of the invention, the amplified RNA molecules are separated by mass and/or charge, such as, for example, via electrophoresis, and they may or may not be denatured during the electrophoresis. Although conventionally RNA is run on a denaturing agarose gel because of the single-stranded nature and secondary structure of the RNA, in particular embodiments the distinguishing RNA pattern is suitably identified on a non-denaturing gel (FIG. 2B). In specific embodiments, the RNA pattern is detected on a RNA microfluidic chip (FIG. 2C). In other specific embodiments, the RNA can be detected using a DNA microfluidics chip.

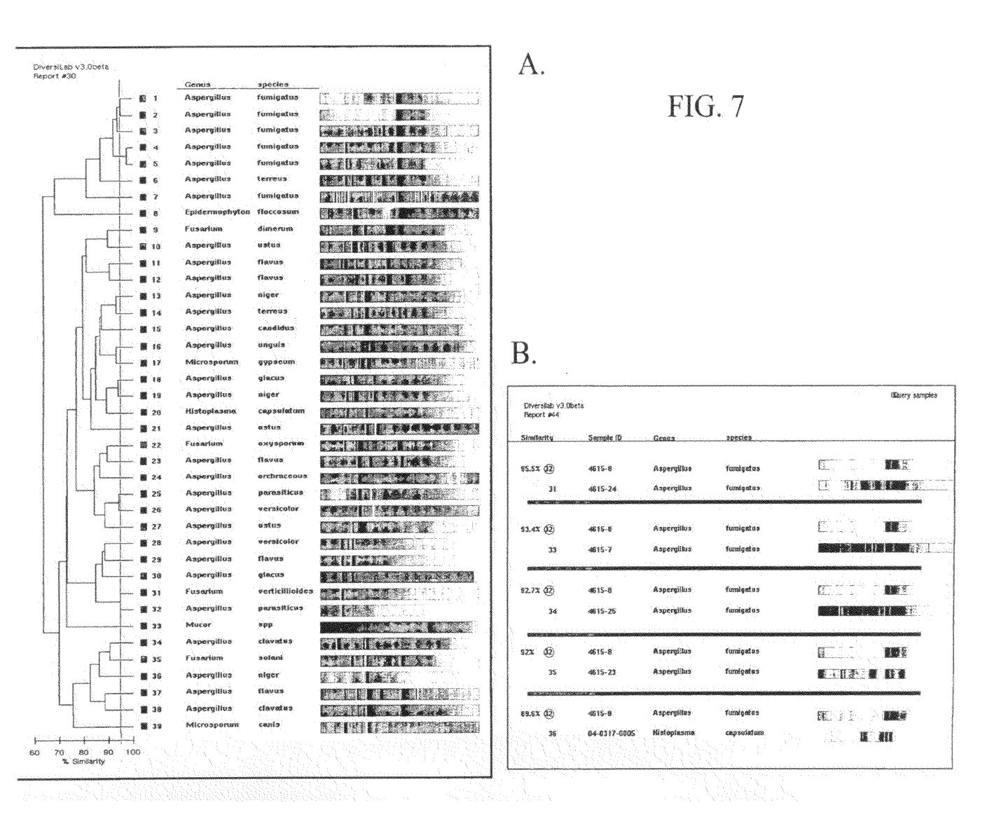

Patterns for detection may be compared to each other or to those in a reference document, database, and/or other known organisms, for example (FIG. 7B).

In some embodiments of the invention, the products are separated, for example on by gel or capillary electrophoresis, by chromatography, by mass spectrometry, or other methods or techniques. The sizing pattern may be determined by an automatic reader, and each pattern can be recognized by a computer means. The reader means will depend on the type of separation which is being used. For instance a wavelength densitometer reader or a fluorescence reader can be used depending on the label being detected. A radioisotope detector can be used for radioisotope labeled primers. In mass spectrometry the ions are detected in the spectrometer. A gel can be stained and read with a densitometer. In specific embodiments, the computer stores fingerprints of known organisms for comparison with test results. In an automated method, bar code readers, laser readers, digitizers, photometers, fluorescent readers and/or computer planimetry can be used to help automate the system. Thus, the separation and reading of the samples can be used to interpret the results and output the data.”

### VII. Kits for Detecting Unknown Organisms

In one embodiment of the present invention, a kit is provided for detecting one or more organisms using the inventive methods and compositions pursuant to the invention. Although the organism may be of any kind, in particular aspects of the invention, the kit is directed to detecting bacteria, fungus, or both. In further embodiments, the kits are employed for distinguishing different genus, species, or sub-species of particular organisms.

In particular, the kit may comprise one or more of the following: one or more primers that target a repetitive sequence and comprises an RNA polymerase site; one or more primers that target a repetitive sequence and lack an RNA polymerase site; a DNA-dependent DNA polymerase; a RNA-dependent DNA polymerase; deoxynucleotides; ribonucleotides; RNaseH; a DNA-dependent RNA polymerase; a reference guide for recognizing a particular RNA pattern indicative of one or more organisms; one or more suitable buffers; water, such as nuclease-free water; or reagents and/or equipment for detection of the RNA, such as for an agarose gel (denaturing or non-denaturing), an RNA microfluidic chip; or a DNA microfluidic chip.

The components of the kit will be housed in a suitable container and may be compartmentalized and/or sterilized. The package may be air-tight and/or water-tight. Particular reagents may be packaged suitably in vials, syringes, packets, bottles, boxes, containers, and so forth. Reagents may be packaged as liquids or as dry components, such as lyophilized components.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Repetitive Amplification Protocol

Although the methods of the present invention allow a variety of parameters to be optimized and still achieve transcription-based amplification originating with repetitive sequence-targeted primers, in specific embodiments the procotol described in this Example is utilized (see FIG. 2B, for example). Although a commercial kit (NucliSens® Basic Kit, bioMerieux, N.C., USA) was modified and employed in this particular study, all reagents are standard in the art and may be provided separately.

1) A Reconsitute Reagent comprises a Reagent Sphere (1.0 sphere) and a Reagent Diluent. The Reagent Sphere includes the following: ribonucleotides (rATP, rUTP, rCTP, rGTP); deoxynucleotides (dATP, dTTP, dGTP, dCTP); ITP; DTT; MgCl₂. EDTA. The Reagent Diluent (approximately 80.0 μL) comprises DMSO and Tris/HCL.

The Reconstitute Reagent is incubated in 50° C. water bath for approximately 25 min. Following a brief vortex at ½ speed, the tube is tapped or flicked to bring all of the reagents to the bottom.

2) A Reconstituted Enzyme comprises an Enzyme Sphere (1.0 sphere) and Enzyme Diluent. The Enzyme Sphere includes AMV-RT, T7 polymerase, and Ribonuclease H. The Enzyme Diluent (approximately 55.0 μL) comprises Sorbitol. Following incubation at room temperature for about 25 minutes, the tubes are briefly vortexed at ½ speed. The tube is tapped or flicked to bring all reagents to the bottom.

3) A KCL/Water Mix (80mM) is prepared while waiting for reagent and enzyme to reconstitute that includes 16.2 mL KCl and water (RNase-free) (13.8 mL). The volume can be changed to vary the concentration of KCL. The tube is briefly vortexed and spun down.

4) A Reagent Mix is provided or obtained and comprises Reconstitute Solution (80.0 μL) and a KCl/Water Solution (30.0 μL). The tube is briefly vortexed at ½ speed and tapped or flicked to bring all reagents to the bottom of the tube.

5) Primers are diluted to 20 μM each. Starting from 200 μM stock: 2 μL primer+18 μL RNase-free water are provided. Equal parts of each primer (E2+E2cT7ggg) are mixed for final concentration of 10 μM each. For example, for 10 reactions, the following is mixed: 5 μL E2 at 20 μM+5 μL E2cT7ggg at 20 μM.

6) A tube is prepared for each reaction. Stock DNA (125 ng total) is added to 0.83 μl Primer Mix to the bottom of each tube. The tubes are tapped on the benchtop to bring reagents to the bottom.

7) The tubes comprising the DNA+primer mix are placed in a 95° C. environment, such as a water bath, for denaturation of the DNA for approximately 5 min. The tubes are then placed in a 65° C. environment, such as a water bath, for approximately 2 min. These steps may be performed in a thermal cycler, such as one utilized for polymerase chain reactions, although this method does not use polymerase chain reaction.

8) Without removing from the 65° C. environment, 9.17 μL Reagent Mix is added to each tube. The mix is pipetted up and down. Incubation is resumed as follows: 65° C. for 30 seconds and 37° C. for 2 min.

9) Without removing from the 37° C. environment, about 5 μL enzyme (a mixture of reverse transcriptase, T7 RNA polymerase, and RNAse H, for example) is added to each tube. The mix is pipetted up and down. Incubation is resumed as follows: 37 deg C. for 90 min.

10) The samples may then be immediately frozen until ready to load on gel or chip or used immediately for detection assays, such as on a gel or chip. A standard agarose gel may be employed such as a 2% agarose gel comprising 6g agarose +300 mL TAE Buffer. The gel may be run for about 100 min at 150V.

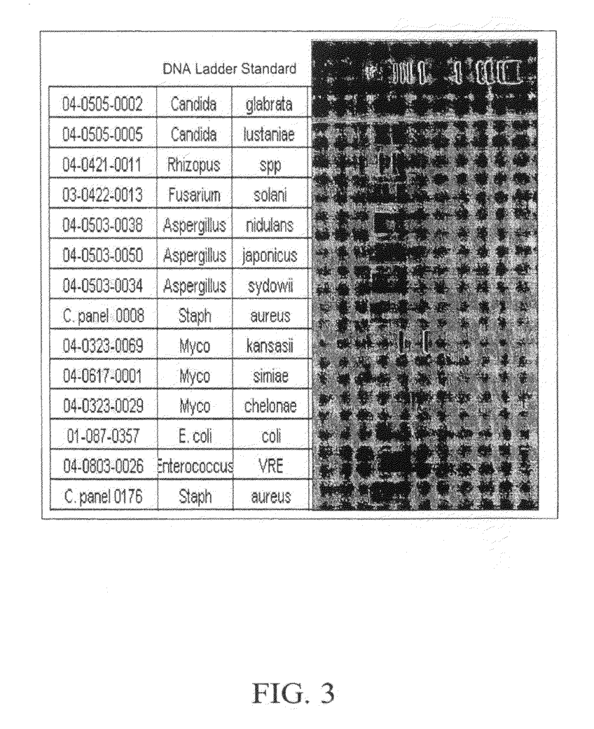

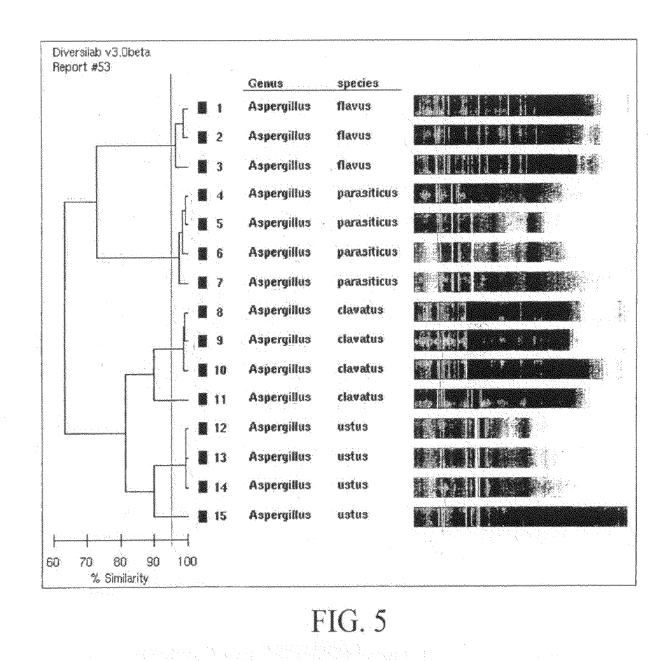

In an alternative embodiment (FIGS. 1 and 3, for example), a single primer is added to the reaction and heat suitable to cause denaturation of dsDNA, such as 95° C.; the heat is reduced and RT enzyme and RT master mix is added for approximately 5 min and a second primer is added for approximately 2 min, if applicable; heat is again re-applied for denaturation; the heat is again reduced and then a mixture of RT, T7 polymerase, and RNAse H plus Reagent mix (comprising deoxynucleotides, DTT, MgCl₂, EDTA, DMSO, Tris/HCL, and KCL); the temperature is further lowered and the Enzyme mix is added. In specific embodiment, these alternative steps comprise the following (illustrated in FIG. 5), wherein a representative sample concentration is about 125 ng total, input primer concentration was about 20 μM total, and KCl was at 80 mM:

1) A mixture of about 7 μl DNA and 0.415 μl E2 or 0.415 μl E2cT7ggg Primer is subjected to about 95° C. for about 2 min. The mixture is exposed to 41° C. for about 4 min, 55° C. for about 7 min, or 60° C. for about 12 min.

2) About 1 μl of RT and 4 μl of RT buffer/nucleotide mix is added after about 2 min at 41° C., 2 min at 55° C., or 2 min at 60° C., followed by a 95° C. approximately 2 min incubation.

3) About 0.415 μl E2 or 0.415 μl E2cT7ggg primer is added, and the reaction is subjected to 95° C. for about 1 min and then 65° C. for about 2 min.

4) About 9.17 μl of the Reaction Solution is heated to 65° C. for about 30 sec and then 37° C. for about 2 min.

5) About 3 μl enzyme is added and the mixture is subjected to 37° C. for about 90 min.

FIG. 3 provides additional data utilizing methods of the invention. In the study demonstrated on the left panels, 7 μl of diluted DNA was added to 1 μl of E2cTag (8.3 μM), which was denatured at 95° C. for 2 min and brought to 65° C. for 2 min. Five μl of RT mix was added, and the mixture was incubated at 65° C. for 2 min. One μL of E2 primer (8.3 μM) was added to the mixture, which was then incubated at 65° C. for 2 min, followed by 95° C. for 1 min and 65° C. for 2 min. Following this, 9.17 μl of reagent solution was added and the mixture was incubated at 65° C. for 30 sec and held at 37° C. for 2 min. Finally, 3 μl of enzyme solution (RT, T7 RNA polymerase, and RNAse H) was added to the mixture, which was then incubated at 37° C. for 90 min. The gel image illustrating ethidium bromide-stained RNA molecules demonstrates that there are differentiating banding patterns at least for different genera and different species within a genus.

FIG. 4 demonstrates banding patterns for various bacterial and fungal isolates using methods of the present invention, including detection of the RAmp amplified product from Candida and Aspergillus using RNA chips (FIG. 4A) showing genus and species discrimination. In addition, RAmp amplified product from Gram+ and Gram− bacteria using DNA chips (FIG. 4B) shows genus, species, and strain (P. aeruginosa) discrimination.

FIG. 5 provides an illustration of species identification among Aspergillus organisms and reproducibility of the fingerprint patterns, as isolates were processed from culture to analysis in triplicate.

### Example 2

### Discrimination of Subspecies and Strains Using the Inventive Methods

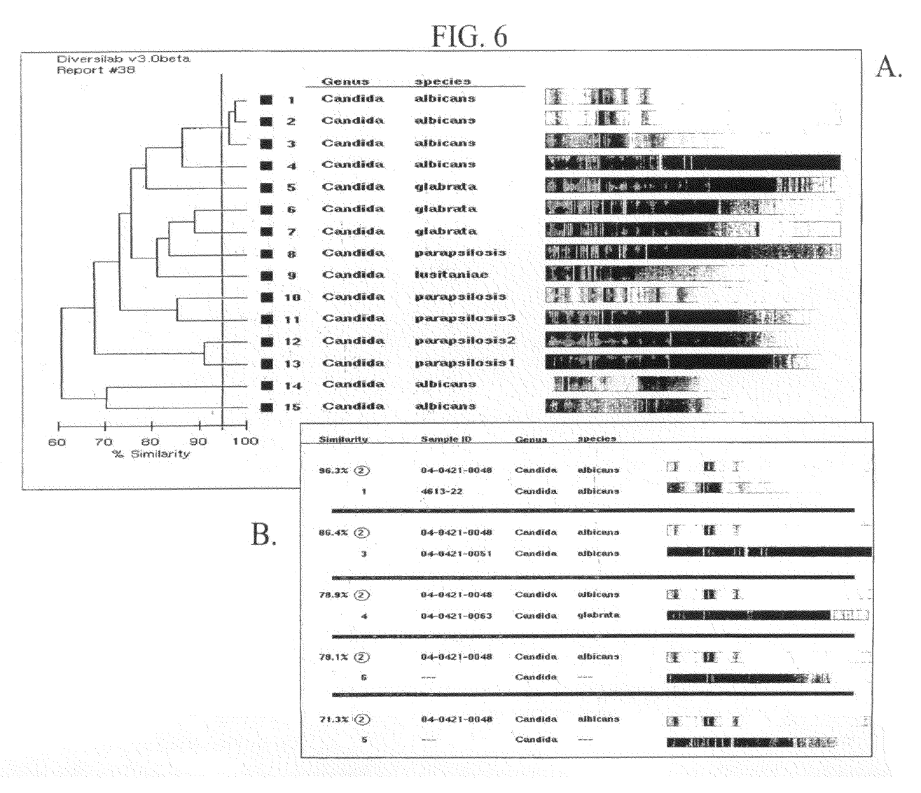

FIG. 6B demonstrates that methods of the invention are sensitive enough to discriminate between subspecies of organisms (for example, Candida parapsilosis 1, Candida parapsilosis 2, and Candida parapsilosis 3). They are also sensitive enough to distinguish between strains (Candida albicans in group 1, 2, 3, and group 14 and 15, for example). Strain differentiation can also be identified for bacteria (FIG. 4B) and fungi such as Aspergillus fumigatus species in FIG. 7A (lanes 1-5) and for A. flavus (lanes 11 and 12), for example.

### Example 3

### Identification of Fungi Using the Inventive Methods

The differentiation between different exemplary fungi is provided in FIG. 7, wherein Aspergillus, Fusarium, Zygomycetes, dimorphic fungi, and Dermatophytes are represented. Identification (FIG. 7B) for Aspergillus fumigatus and identification of C. albicans (FIG. 6B) are shown by pattern matching using a characterized library.

### Example 4

### Identification of an Organism

In particular embodiments, the methods and compositions of the present invention are utilized to identify one or more organisms from a sample. In this exemplary embodiment, one or more organisms is obtained, such as from a source suspected of contamination.

DNA is obtained from the one or more organisms from the sample. For dsDNA embodiments, the dsDNA is denatured, and a primer comprising DNA-dependent RNA polymerase promoter sequence or the complement thereof and sequence that targets one or more repetitive sequences in the organism hybridizes to the corresponding sequence on the DNA molecule. A DNA polymerase extends the primer such that upon polymerization through the promoter site sequence or complement thereof, the corresponding complement of the promoter site sequence or promoter site sequence is generated.

The DNA-dependent RNA polymerase may begin generating RNA molecules from the promoter site, further defined as doing so by transcription. The RNA molecules may be further utilized as templates for making dsDNA and one or more rounds of RNA amplification. In particular embodiments, the RNA molecules are distributed on an agarose gel or microfluidics chip, and the pattern of the RNA molecules determines the organism from which the original DNA was obtained.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PATENTS AND PATENT APPLICATIONS

U.S. Pat. No. 5,523,217

U.S. Pat. No. 5,691,136

U.S. Pat. No. 6,074,820

U.S. Pat. No. 6,251,639

U.S. Pat. No. 6,686,156

U.S. Pat. No. 6,692,918

WO 96/02668

WO 99/25868

WO 99/51771

### PUBLICATIONS

Antonio, M. A. D. and Hillier, S. L. 2003. DNA fingerprinting of Lactobacillus crispatus strain CTV-05 by repetitive element sequence-based PCR analysis in a pilot study of vaginal colonization. J. Clin. Microbiol. 41(5):1881-1887.
Aranda-Olmedo, I., Tobes, R., Manzanera, M., Ramos, J. L., and Margques, S. 2002. Species-specific repetitive extragenic palindromic (REP) sequences in Pseudomonas putida. Nucl. Acids Res. 30(8):1826-1833.
Bouzar, H., J. B. Jones, R. E. Stall, F. J. Louws, M. Schnieder, J. L. W. Rademaker, F. J. de Bruijn, and L. E. Jackson. 1999. Multiphasic analysis of xanthomonads causing bacterial spot disease on tomato and pepper in the Caribbean and Central America: evidence for common lineages within and between countries. Phytopathology 89:328-335.
Bricker, B. J., Ewalt, D. R., and Halling, S. M., Brucella ‘HOOF-Prints’: strain typing by multi-locus analysis of variable number tandem repeats (VNTRs). BMC Microbiolog. 2003, 3:15.
Buchko, J. and G. R. Klassen. 1990. Detection of length heterogeneity in the ribosomal DNA of Pythium ultimum by PCR amplification of the intergenic region. Curr. Genet. 18:203-205.
de Bruijn, F. J. 1992. Use of repetitive (repetitive extragenic palindromic and enterobacterial repetitive intergeneric consensus) sequences and the polymerase chain reaction to fingerprint the genomes of Rhizobium meliloti isolates and other soil bacteria. Appl. Environ. Microbiol. 58:2180-2187.
Dombek, P. E., Johnson, L. K., Zimmerley, S. T., and M. J. Sadowsky. Use of repetitive DNA sequences and the PCR to differentiate E. coli isolates from human and animal sources. Applied Environ. Microbiol. 66(6):2572-2577.
Go, M. F., Chan, K. Y., Versalovic, J., Koeuth, T., Graham, D. Y., and J. R. Lupski. 1995. Cluster Analysis of Helicobacter pylori Genomic DNA fingerprints suggests gastroduodenal disease-specific associations. Scand. J. Gastroenterol. 30:640-646.
Johnson, J. R., Clabots, C., Azar, M., Boxrud, D. J., Besser, J. M., and J. R. Thurn. 2001. Molecular analysis of a hospital cafeteria-associated Salmonellosis outbreak using modified repetitive elements PCR fingerprinting. J. Clin. Microbiol. 39(10):3452-3460.
Judd, A. K., M. Schneider, M. J. Sadowsky, and F. J. de Bruijn. 1993. Use of repetitive sequences and the polymerase chain reaction technique to classify genetically related Bradyrhizobium japonicum serocluster 123 strains. Appl. Environ. Microbiol. 59:1702-1708.
Kanaya E, Uchiyama Y, Ohtsuka E, Ueno Y, Ikehara M, and Kanaya S. 1994. Kinetic analyses of DNA-linked ribonucleases H with different sizes of DNA. FEBS Lett. November 7; 354(2):227-31.
Li W H, Gu Z, Wang H, Nekrutenko A. Evolutionary analyses of the human genome. Nature. 2001 Feb. 15; 409(6822):847-9.
Lipman, L. J. A., A. de Nijs, T. J. G. M. Lam, and W. Gaastra. 1995. Identification of Escherichia coli strains from cows with clinical mastitis by serotyping and DNA polymorphism patterns with REP and ERIC primers. Vet. Microbiol. 43:13-19.
Makalowski, W. “SINEs as a genomic scrap yard: an essay on genomic evolution”, In “The Impact of Short Interspersed Elements (SINEs) on the Host Genome” (ed R J Maraia), pp. 81-104, (R.G. Landes Company, Austin, 1995.
Miehlke, S., Thomas, R., Guiterrez, O., Graham, D. Y., and M. F. Go. 1999. DNA fingerprinting of single colonies of Helicobacter pylori from gastric cancer patients suggests infection with a single predominant strain. J. Clin. Microbiol. 37(1):245-247.
Riley D E, Samadpour M, Krieger J N. 1991. Detection of variable DNA repeats in diverse eukaryotic microorganisms by a single set of polymerase chain reaction primers. J Clin Microbiol., 29(12):2746-51.
Sadowsky, M. J., L. L. Kinkel, J. H. Bowers, and J. L. Schottel. 1996. Use of repetitive intergenic DNA sequences to classify pathogenic and disease-suppressive Streptomyces strains. Appl. Environ. Microbiol. 62:3489-3493.
Schonian, G., Schweynoch, Crola, Zlateva, K., Oskam, L., Kroon, N., Graser, Y., Presber, W. 1996. Identification and determination of the relationships of species and strains within the genus Leishmania using single primers in the polymerase chain reaction. Molec. Biochem. Parasitology. 77:19-29.
Versalovic, J., Koeuth, T., and J. R. Lupski. 1991. Distribution of repetitive DNA sequences in eubacteria and application to fingerprinting of bacterial genomes. Nucl. Acids Res. 19(24):6823-6831.
Versalovic, J., F. J. de Bruijn, and J. R. Lupski. 1998. Repetitive sequence-based PCR (rep-PCR) DNA fingerprinting of bacterial genomes, p. 437-454. In F. J. de Bruijn, J. R. Lupski, and G. M. Weinstock (ed.), Bacterial genomes: physical structure and analysis. Chapman and Hall, New York, N.Y.
Voisset, C., Mandrand, B., and Paranhos-Baccala, G. 2000. RNA amplification technique, NASBA, also amplifies homologous plasmid DNa in non-denaturing conditions. BioTechn. 29:236-240.
Yamamura H, Hayakawa M, Nakagawa Y, Iimura Y. 2004. Characterization of Nocardia asteroides isolates from different ecological habitats on the basis of repetitive extragenic palindromic-PCR fingerprinting. Appl Environ Microbiol. May; 70(5):3149-51.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1.-22. (canceled)

23. A method of identifying an organism having two or more repetitive DNA sequences, comprising the steps of:
providing at least one ds DNA polynucleotide from the organism, wherein said polynucleotide comprises the two or more repetitive sequences;
providing at least a first primer that hybridizes to one or more repetitive sequences in the polynucleotide;
amplifying at least part of the polynucleotide under conditions that produce RNA molecules; and
identifying the organism based on a characteristic pattern from said molecules.

24. The method of claim 23, wherein the amplifying step is further defined as comprising the steps of:
producing a double stranded DNA polynucleotide comprising at least part of one or more of the repetitive sequences and a DNA-dependent RNA polymerase promoter sequence; and
polymerizing the RNA molecules with a DNA-dependent RNA polymerase.

25. The method of claim 23, wherein the organism is a fungus, a bacteria, a mammal, an insect, a marine organism; reptile, plant, or virus.

26. The method of claim 23, wherein the identifying step comprises electrophoresis of said RNA molecules.

27. A kit housed in a suitable container, comprising one or more of the following:
at least one primer that targets a repetitive sequence;
buffer;
ribonucleotides;
deoxyribonucleotides;
RNA-digesting enzyme;
DNA-dependent DNA polymerase;
RNA-dependent DNA polymerase; and
DNA-dependent RNA polymerase.

28. The kit of claim 27, wherein the primer is further defined as comprising a DNA-dependent RNA polymerase promoter site or the complement thereof.

29. The kit of claim 28, wherein the RNA polymerase promoter site is further defined as a T7 RNA polymerase promoter site, Thermus Thermostable RNA Polymerase, or a mixture thereof.

30.-35. (canceled)

36. A pattern of RNA molecules indicative of an organism, said organism comprising a DNA polynucleotide having two or more repetitive sequences, wherein the pattern is produced by the separation of the RNA molecules based on their charge, their size, their secondary structure, or a combination thereof, wherein at least the majority of the RNA molecules comprise at least one sequence derived from a repetitive sequence of the organism.

37. The pattern of claim 36, wherein a sequence derived from a repetitive sequence of the organism is located at the 5′ end, the 3′ end, or both.

38. The pattern of claim 36, further defined as being identified in a sequence-independent manner.

39. The pattern of claim 36, further defined as being comprised in a matrix.

40. The pattern of claim 39, wherein the matrix is a gel, a chip, an electropherogram, a paper, or a microarray.

41. The method of claim 23, further providing a second primer comprising sequence that hybridizes to at least part of a repetitive sequence.

42. The method of claim 41, wherein the first and second primers hybridize to substantially the same repetitive sequence.

43. The method of claim 41, wherein the first and second primers hybridizes to different repetitive sequences.

44. The method of claim 23, wherein the identifying step comprises electrophoresis, microfluidics chip analysis, or a combination thereof.
